(19) United States
(12) Patent Application Publication
(10) Pub. No.: US 2025/0025639 A1
(43) Pub. Date: Jan. 23, 2025

(54) DEVICES, SYSTEMS, AND METHODS FOR DELIVERY OF MULTI-COMPONENT SPACERS AND HYDRO-DISSECTION SOLUTIONS

(71) Applicant: Bard Peripheral Vascular, Inc., Franklin Lakes, NJ (US)

(72) Inventors: Dylan Beyhl, Scottsdale, AZ (US); Jordan Addison, Gilbert, AZ (US); Heather Storm, Phoenix, AZ (US); Luke Winterstein, Tempe, AZ (US); Nick Mowrey, Scottsdale, AZ (US)

(73) Assignee: Bard Peripheral Vascular, Inc., Franklin Lakes, NJ (US)

(21) Appl. No.: 18/712,812

(22) PCT Filed: Nov. 23, 2021
(86) PCT No.: PCT/US2021/060570

§ 371 Date: May 23, 2024

(51) Int. Cl. A61M 5/32; A61M 5/19

(52) U.S. Cl. CPC A61M 5/3293; A61M 5/19; A61M 5/3286

(57) ABSTRACT

Delivery devices, as well as systems incorporating the same and methods of using the same are disclosed. A needle assembly includes a hub having first and second input ports and an auxiliary port, and an injection needle assembly. The injection needle assembly includes an elongate hollow stylet extending distally from the hub that includes an outer side wall defining an outer lumen fluidly coupled to the auxiliary port, a first inner side wall defining a middle lumen disposed within the outer lumen that is fluidly coupled to the first input port, a second inner side wall defining an inner lumen disposed within the middle lumen that is fluidly coupled to the second input port, and a mixing chamber fluidly coupled to the outer lumen, the middle lumen, and the inner lumen.

### BACKGROUND

### Field

The present disclosure generally relates to component delivery systems, and more specifically, to devices, systems, and methods for delivering a plurality of components.

### Technical Background

Prostate cancer is the most common non-skin cancer diagnosed in men. Radiation therapy is an excellent treatment option for prostate cancer. However, radiation exposure can cause bowel side effects.

Hydrogel spacers can be used to reduce or minimize bowel injury by providing a space between the bowel (rectum) and the prostate. In addition, hydrogel spacers can also be used to reduce or minimize urinary and sexual symptoms.

However, when placing such spacers, sometimes a need arises to hydro-dissect the space prior to placement of the spacer. This step typically requires additional components or devices to perform the dissection separate from the apparatus that delivers the spacer. In addition, this step usually involves additional punctures and/or insertions that can result in increased complications.

### SUMMARY

In one aspect, a needle assembly includes a hub having first and second input ports and an auxiliary port, and an injection needle assembly. The injection needle assembly includes an elongate hollow stylet extending distally from the hub. The elongate hollow stylet includes a proximal portion at the hub and a distal portion spaced a distance from the proximal portion. The elongate hollow stylet further includes an outer side wall extending from the proximal portion to the distal portion and defining an outer lumen that is fluidly coupled to the auxiliary port. The elongate hollow stylet further includes a first inner side wall extending from the proximal portion to the distal portion and defining a middle lumen disposed within the outer lumen such that the outer lumen surrounds the middle lumen. The middle lumen is fluidly coupled to the first input port. The elongate hollow stylet further includes a second inner side wall extending from the proximal portion to the distal portion and defining an inner lumen disposed within the middle lumen such that the middle lumen surrounds the inner lumen. The inner lumen is fluidly coupled to the second input port. The elongate hollow stylet further includes a mixing chamber disposed at the distal portion of the elongate hollow stylet. The mixing chamber is fluidly coupled to the outer lumen, the middle lumen, and the inner lumen.

In another aspect, a fluid delivery system includes a dual chamber applicator having two chambers separate from one another, each chamber including at least one output port on a distal end thereof. The fluid delivery system further includes an injection needle assembly. The injection needle assembly includes a hub having first and second input ports aligned with the at least one output port of each of the two chambers of the dual chamber applicator, and an auxiliary port. The injection needle assembly further includes an elongate hollow stylet extending distally from the hub. The elongate hollow stylet includes a proximal portion at the hub and a distal portion spaced a distance from the proximal portion. The elongate hollow stylet further includes an outer side wall extending from the proximal portion to the distal portion and defining an outer lumen that is fluidly coupled to the auxiliary port. The elongate hollow stylet further includes a first inner side wall extending from the proximal portion to the distal portion and defining a middle lumen disposed within the outer lumen such that the outer lumen surrounds the middle lumen. The middle lumen is fluidly coupled to the first input port. The elongate hollow stylet further includes a second inner side wall extending from the proximal portion to the distal portion and defining an inner lumen disposed within the middle lumen such that the middle lumen surrounds the inner lumen. The inner lumen is fluidly coupled to the second input port. The elongate hollow stylet further includes a mixing chamber disposed at the distal portion of the elongate hollow stylet. The mixing chamber is fluidly coupled to the outer lumen, the middle lumen, and the inner lumen.

In yet another aspect, a fluid delivery system includes an injection needle assembly having a proximal end and a distal end spaced a length from the proximal end. The injection needle assembly includes a hub disposed at the proximal end. The hub includes a first input port fluidly coupled to a first lumen extending the length of the injection needle assembly, a second input port fluidly coupled to a second lumen extending the length of the injection needle assembly, the second lumen separated from the first lumen, and an auxiliary port fluidly coupled to a third lumen extending the length of the injection needle assembly, the third lumen separated from the first lumen and the second lumen. The fluid delivery system further includes a dual chamber applicator having a first chamber with a first output port aligned with the first input port of the hub, a second chamber separate from the first chamber, the second chamber having a second output port aligned with the second input port of the hub; and an auxiliary applicator fluidly coupled to the auxiliary port of the hub.

In yet another aspect, a method of delivering fluids to a subject includes inserting a distal end of an injection needle assembly of a fluid delivery system into a target area of the subject. The fluid delivery system comprises a dual chamber applicator and an auxiliary applicator fluidly coupled via a hub and an elongate hollow stylet defining three separate fluid paths to a distal mixing chamber disposed at the end distal portion of the elongate hollow stylet. The method further includes causing a first fluid within the auxiliary applicator to be delivered to the target area via a first one of the three separate fluid paths to create a space. The method further includes causing two precursor materials within the dual chamber applicator to be delivered to the space via a second and a third one of the three separate fluid paths and mixed. Mixing of the two precursor materials results in a hydrogel being formed in the space.

Additional features and advantages of the aspects described herein will be set forth in the detailed description which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 depicts a side view of an illustrative multi-component delivery system including a dual chamber applicator, an auxiliary applicator, and an injection needle assembly with a hub having input ports and an auxiliary port and an elongate hollow stylet extending from the hub assembly according to one or more aspects shown and described herein;

FIG. 2 depicts a perspective view of the injection needle assembly of FIG. 1 according to one or more aspects shown and described herein;

FIG. 3A depicts a side view of an illustrative elongate hollow stylet according to one or more aspects shown and described herein;

FIG. 3B depicts a front view of the elongate hollow stylet of FIG. 3A according to one or more aspects shown and described herein;

FIG. 4 depicts a section view of a distal portion of an illustrative injection needle assembly having a sharp tip according to one or more aspects shown and described herein;

FIG. 5 depicts a section view of a distal portion of another illustrative injection needle assembly having a blunt tip and radially disposed outlet ports according to one or more aspects shown and described herein;

FIG. 6 depicts a section view of a distal portion of another illustrative injection needle assembly having a blunt tip and a distally disposed outlet port according to one or more aspects shown and described herein; and

FIG. 7 depicts a quarter cutaway view of the hub of the injection needle assembly according to one or more aspects shown and described herein.

### DETAILED DESCRIPTION

The present disclosure, in one form, is related to multi-component hydrogel precursor delivery devices that incorporate an auxiliary port for delivery of a hydro-dissection solution prior to precursor component delivery, as well as systems and methods that incorporate the same. The delivery systems described herein include components that deliver the precursor materials for the multicomponent hydrogel, which includes a dual chamber applicator. In addition, the delivery systems described herein further include an injection needle assembly and an auxiliary syringe used to deliver the hydro-dissecting solution. Since the precursor materials located in the dual chamber applicator quickly form the hydrogel spacer when combined together, it is necessary to ensure the combination occurs at the side where the hydrogel product is to be distributed. Further, it is sometimes necessary to perform hydro-dissection at the site prior to delivery of the hydrogel spacer, but use of separate components can result in additional punctures or insertions, which can increase a chance of complications. As such, the devices, systems, and methods described herein include a hub coupled to an elongate hollow stylet, where the hub includes a pair of input ports for coupling to the dual chamber applicator and an auxiliary port for coupling to the auxiliary syringe, and the elongate hollow stylet includes a mixing chamber at a distal end thereof, an inner lumen disposed within a middle lumen that is disposed within an outer lumen to define three separate passageways, two of which extend from the two chambers of the applicator and the third from the auxiliary port to the mixing chamber at the distal end.

Hydrogel spacers are an absorbable gel that temporarily creates a space between the prostate and the rectum. The extra space protects the rectum from radiation exposure during treatment. In addition, the spacer can reduce the harmful effects of radiation therapy, allow for improved targeting, allow for higher doses of radiation, and allowing for a shorter treatment time.

A typical spacer placement procedure includes placing a needle between the prostate and the rectum, hydro-dissecting the space by injecting saline or another hydro-dissecting solution, removing the needle, placing an applicator within the space, and placing the precursor materials, which solidify into the gel spacer. The spacer is left in the space throughout an entire treatment period, and is eventually absorbed by the body after a period of time has elapsed (e.g., 6 months).

An advantage of the present disclosure is that the various aspects described herein improve upon typical solutions in that the multi-component hydrogel spacer is formed from the hydrogel components at a location where it is to be placed. Further, another advantage of the present disclosure is the hydro-dissection solution can be delivered without the need for performing additional punctures and/or insertions.

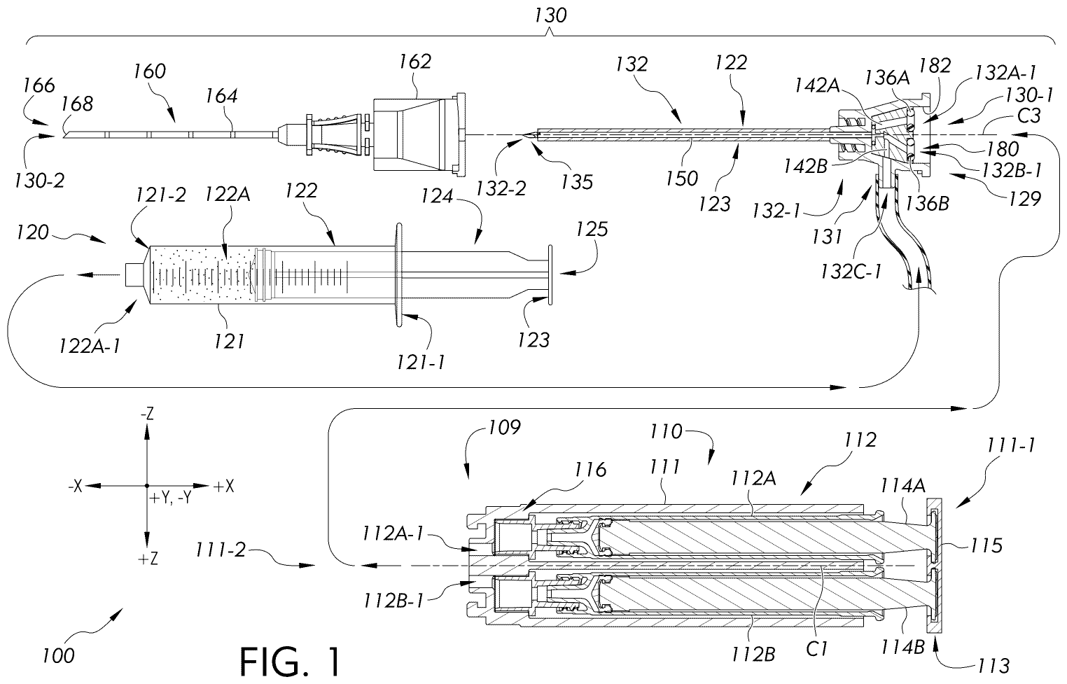

Turning now to the drawings, FIG. 1 depicts an illustrative delivery system 100 according to various embodiments. The delivery system 100, in accordance with an aspect of the present invention, may be for use in a spacer delivery procedure whereby a multi-component spacer (e.g., a hydrogel) is delivered to a site that has been prepped via a hydro-dissection procedure. The delivery system 100 generally includes a dual chamber applicator 110, an auxiliary applicator 120, and/or an injection needle assembly 130. The various components for the delivery system 100 are couplable together for the purposes of delivering a dual component mixture (e.g., a dual component hydrogel) and an additional material (e.g., saline or other hydro-dissection solution, or the like), as indicated by the dashed lines in FIG. 1 and described herein.

When the dual chamber applicator 110 is coupled to the injection needle assembly 130, the delivery system 100 facilitates delivery of flowable hydrogel precursor materials to an injection site. The hydrogel precursor materials may be injected when the needle portion of the injection needle assembly 130 is located in a target site, such as a space between the rectum and the prostate of a subject that is formed by prior injection of a hydro-dissection solution via the injection needle assembly 130. It is noted that while the components described herein are specifically described with respect to a procedure that involves placing a spacer between the rectum and the prostate, other uses are contemplated and included within the scope of the present application.

The dual chamber applicator 110 generally includes a body 111 having a proximal end 111-1 and a distal end 111-2 spaced a distance apart from the proximal end 111-1. The body 111 also defines a pair of syringes 112. The dual chamber applicator 110 is configured to separately carry each of a first precursor material of the multi-component hydrogel and a second precursor material of the multi-component hydrogel. Illustrative examples of first precursor materials include, but are not limited to, albumin, polyethylenimine (PEI), an amine containing polyethylene glycol (PEG) or protein, or the like. Illustrative examples of second precursor materials include, but are not limited to, an N-hydroxysuccinimide (NHS) ester component such as PEG-(SS)2, PEG-(SS)4, PEG-(SS)8, PEG-(SG)4, PEG-(SG)8, and/or the like. In some embodiments, molecular weights of the PEG components may range from about 2,000 to about 100,000. In the present embodiment, the first precursor material and the second precursor material are combined and mixed within a mixing chamber of the injection needle assembly 130, as will be described in greater detail herein.

The pair of syringes 112 includes an actuator 113, a first component chamber 112A, and a second component chamber 112B. The first component chamber 112A may be, for example, a cylindrical tube that is configured to carry the first precursor material of the hydrogel spacer. The first component chamber 112A has a first output port 112A-1 (e.g., a first component port). The second component chamber 112B also may be, for example, a cylindrical tube that is configured to carry the second precursor material of the hydrogel spacer. The second component chamber 112B has a second output port 112B-1 (e.g., a second component port). In some aspects, the first component chamber 112A and the second component chamber 112B are arranged in a substantially longitudinally parallel arrangement.

In some aspects, the actuator 113 includes a first piston 114A, a second piston 114B, and a handle 115. The handle 115 is in the form of a link member that perpendicularly extends between, and is connected to, each of the first piston 114A and the second piston 114B to facilitate simultaneous movement of the first piston 114A and the second piston 114B with the depression or retraction of the handle 115. The first piston 114A is in the form of a plunger that is positioned in the first component chamber 112A proximal to the first sealant component. The second piston 114B is in the form of a plunger that is positioned in the second component chamber 112B proximal to the second sealant component.

The first output port 112A-1 of the first component chamber 112A and the second output port 112B-1 of the second component chamber 112B may be arranged within the distal end 111-2 of the body 111 of the dual chamber applicator 110. The first output port 112A-1 and the second output port 112B-1 are generally fluid outputs that are aligned with other ports of other components as described herein such that the first and second precursor materials can be dispensed from and/or received within the respective component chambers 112A, 112B. In some aspects, the first output port 112A-1 may be concentrically aligned with the first component chamber 112A and the second output port 112B-1 may be concentrically aligned with the second component chamber 112B. However, in other aspects, such as the aspect depicted in FIG. 1, the first output port 112A-1 may be located radially inward of a central area of the first component chamber 112A and the second output port 112B-1 may be located radially inward of a central area of the second component chamber 112B such that the first output port 112A-1 and the second output port 112B-1 are as close as possible to a center axis C1 of the body 111 of the dual chamber applicator 110 to facilitate alignment with the other components of the delivery system 100 described herein.

The body 111 of the dual chamber applicator 110 generally includes a connection mechanism 109 for connecting the dual chamber applicator 110 to the injection needle assembly 130. For example, in some embodiments, the body 111 of the dual chamber applicator 110 may include a quarter turn connector or other connector integrated with the distal end 111-2 of the dual chamber applicator 110. In some embodiments, various components of the connection mechanism 109 are integrated with the body 111 such that the connection mechanism 109 and the body 111 are a single monolithic piece. However, it should be understood that this is merely illustrative and the various components of the connection mechanism 109 may be separate pieces that are permanently or semi-permanently joined with the body 111 of the dual chamber applicator 110 (e.g., permanently or semi-permanently joined with a distal coupling piece 116 of the dual chamber applicator 110).

The connection mechanism 109 is generally located at the distal end 111-2 of the body 111 of the dual chamber applicator 110 such that various components of the connection mechanism 109 are positioned adjacent to first output port 112A-1 and the second output port 112B-1. The connection mechanism 109 may be generally shaped and sized to releasably interlock with a corresponding connection mechanism 129 of the injection needle assembly 130. As will be described in greater detail herein, when the injection needle assembly 130 is coupled to the dual chamber applicator 110 via the connection mechanisms 109, 129 thereof, the ports thereof are aligned and sealed with the first output port 112A-1 and the second output port 112B-1 of the dual chamber applicator 110.

While the connection mechanism 109 is not limited by the present disclosure and may be any mechanism that provides a connection between dual chamber applicator 110 and the injection needle assembly 130, one illustrative example of the connection mechanism 109 may include a protrusion (e.g., a circular protrusion) extending distally from the distal end 111-2 of the dual chamber applicator 110 and/or one or more coupling members disposed radially outward of the protrusion. The first output port 112A-1 and the second output port 112B-1 may be disposed within the protrusion in such embodiments. That is, the openings into the first component chamber 112A and the second component chamber 112B are located on the protrusion in some embodiments.

The protrusion is generally shaped and sized to correspond to a recess formed in the injection needle assembly 130, as described in greater detail herein. The protrusion may generally be disposed in or around a central area of the distal end 111-2 of the body 111. In some embodiments, the protrusion may be concentric with the body such that the center axis C1 of the body 111 extends through a center of the protrusion. The distance that the protrusion extends away from the distal end 111-2 of the body is generally a distance that corresponds to a depth of the recess formed in the injection needle assembly 130 such that the protrusion can be completely inserted therein, but is otherwise not limited by the present disclosure.

In some embodiments, the connection mechanism 109 may include one or more coupling members. For example, coupling members may extend from the distal end 111-2 of the body 111 of the dual chamber applicator 110 and are generally shaped and sized to retain the injection needle assembly 130 when coupled to the dual chamber applicator 110. Illustrative coupling members include, but are not limited to, a bayonet style coupling member, an L-beam coupling member, or the like.

The auxiliary applicator 120 generally includes a body 121 having a proximal end 121-1 and a distal end 121-2 spaced a distance apart from the proximal end 121-1. The body 121 also defines a syringe 122 The syringe 122 is configured to carry a material, such as a hydro-dissection solution (e.g., saline or another hydro-dissection solution), an anesthetic solution, and/or the like.

The syringe 122 includes an actuator 123 and a chamber 122A. The chamber 122A may be, for example, a cylindrical tube that is configured to carry the hydro-dissection solution, the anesthetic solution, and/or the like. The chamber 122A has an output port 122A-1.

In some aspects, the actuator 123 includes a piston 124 and a handle 125. The handle 125 is connected to the piston 124 at a proximal end thereof to facilitate movement of the piston 124 with the depression or retraction of the handle 125. The piston 124 is in the form of a plunger that is positioned in the chamber 122A proximal to the hydro-dissection solution, the anesthetic solution, and/or the like.

The output port 122A-1 of the chamber 122A may be arranged within the distal end 121-2 of the body 121 of the applicator 120. The output port 122A-1 is generally a fluid output that is fluidly coupled to an auxiliary port 132C-1 of the hub 131 such that the fluid within the chamber 122A is deliverable via the auxiliary port 132C-1 of the hub 131. In some aspects, the output port 122A-1 may be couplable to the auxiliary port 132C-1 via tubing or the like, as depicted in FIG. 1.

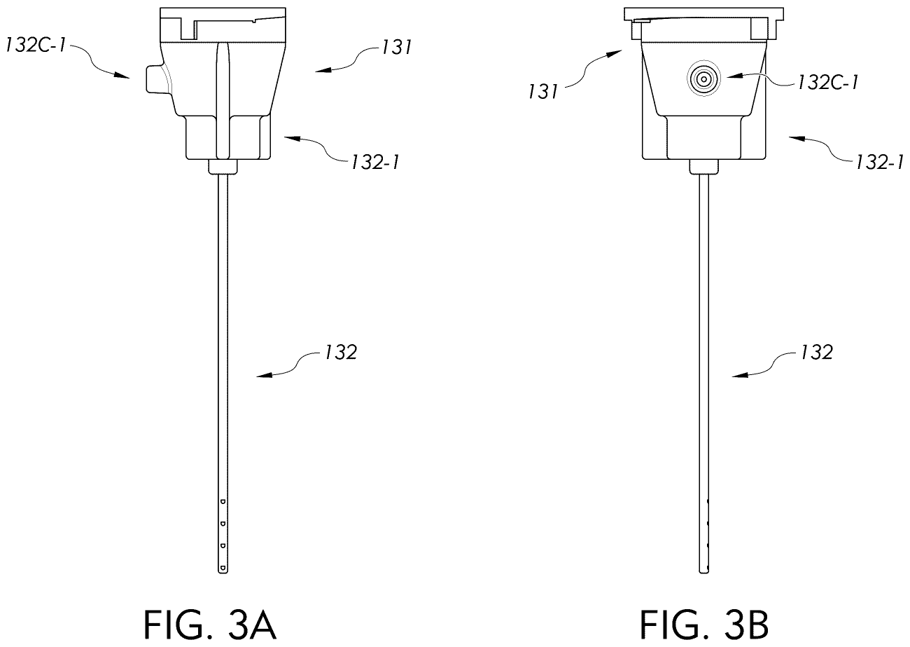

Referring now to FIGS. 1, 2, and 3A-3B, the injection needle assembly 130 has a proximal end 130-1 that extends proximally (e.g., in the +x direction of the coordinate axes of FIG. 1) and a distal end 130-2 that extends distally (e.g., in the −x direction of the coordinate axes of FIG. 1). The injection needle assembly 130 generally includes a hub 131 and an elongate hollow stylet 132 that extends distally (e.g., in the −x direction of the coordinate axes of FIG. 1) from the hub 131. The elongate hollow stylet 132 has a proximal end 132-1 and a distal end 132-2. The hub 131 is fixedly attached (e.g., through overmolding, adhesive and/or pressed fit) to the proximal end 132-1 of the elongate hollow stylet 132. The hub 131 includes a plurality of input ports (e.g., a first input port 132A-1, a second input port 132B-1, and/or the auxiliary input port 132C-1) of the injection needle assembly 130. The hub 131 is configured for removable connection to the dual chamber applicator 110 via the corresponding connection mechanism 129 such that, when connected, the first input port 132A-1 of the injection needle assembly 130 is aligned and sealed with the first output port 112A-1 of the dual chamber applicator 110 and the second input port 132B-1 of the injection needle assembly 130 is aligned and sealed with the second output port 112B-1 of the dual chamber applicator 110, as described in greater detail herein. In addition, the hub 131 for fluid coupling to the applicator 120 via the auxiliary input port 132C-1 (e.g., via tubing or the like coupled between the output port 122A-1 of the chamber 122A of the syringe 122 and the auxiliary input port 132C-1 of the hub 131).

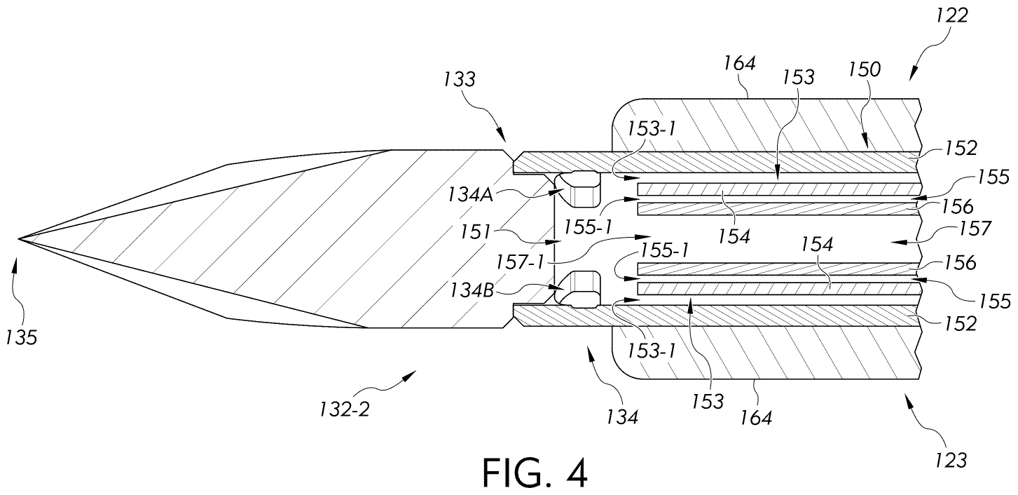

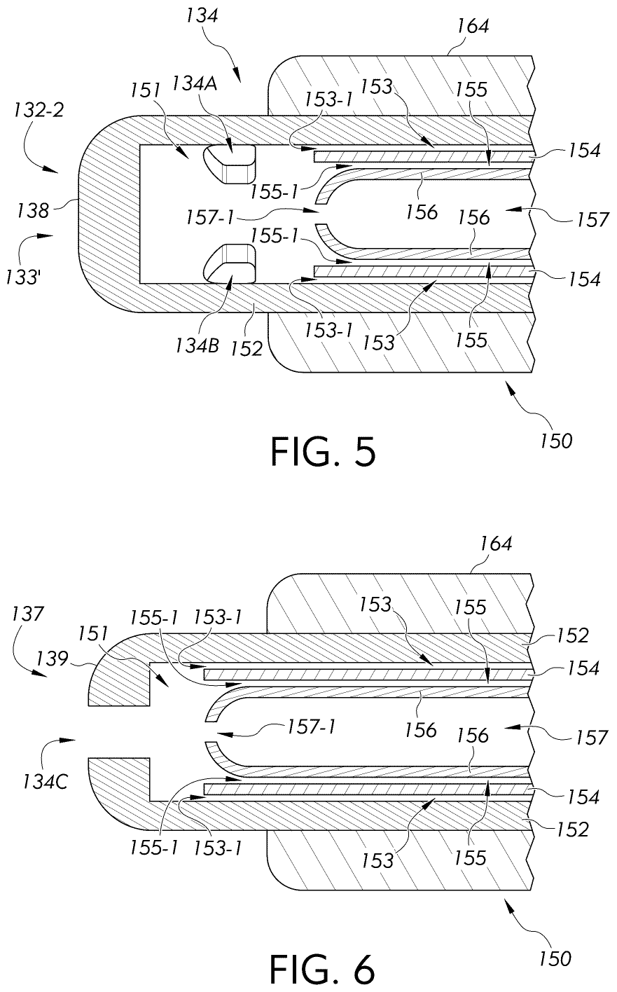

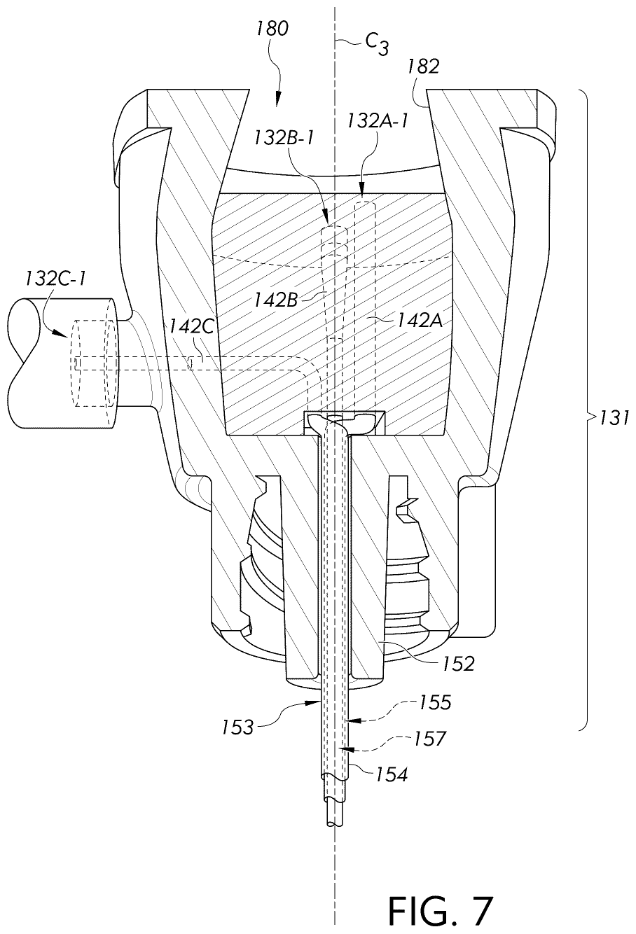

The elongate hollow stylet 132 of injection needle assembly 130 is configured to facilitate fluid communication with the plurality of output ports 112A-1, 112B-1 of the dual chamber applicator 110 so as to receive the two precursor materials of the hydrogel spacer from dual chamber applicator 110 and direct the two components to the distal end 132-2 thereof for mixing and delivery. In addition, the elongate hollow stylet 132 of the injection needle assembly is configured to facilitate fluid communication with the output port 122A-1 of the auxiliary applicator 120 so as to receive the contents of the auxiliary applicator 120 and direct the contents to the distal end 132-2 thereof for delivery. Referring briefly to FIGS. 4-6, the distal end 132-2 may include a closed distal end 133, 133′ (FIGS. 4 and 5) or an open distal portion 137 (FIG. 6). In addition, the distal end 132-2 may include a plurality of side ports 134 (e.g., two, three, or more) proximal to the closed distal end 133, 133′ (FIGS. 4 and 5) or a distal port 134C (FIG. 6, also referred to as a tip port). In the embodiment of FIG. 4, the closed distal end 133 of the elongate hollow stylet 132 may be, for example, a closed stylet needle tip 135. In the embodiment of FIG. 5, the closed distal end 133′ of the elongate hollow stylet 132 may be, for example, a blunt tip that avoids or reduces the chance of puncture of structures (e.g., rectal wall). Similarly, in the embodiment of FIG. 6, the open distal portion 137 of the elongate hollow stylet 132 may be, for example, a blunt tip that avoids or reduces the chance of puncture of structures (e.g., rectal wall).

Referring to FIG. 1 and with reference to the embodiment of FIG. 4, the elongate hollow stylet 132 may be constructed, for example, by an elongate cannula 150 being fixedly connected to the closed stylet needle tip 135. As particularly shown in FIG. 4, the plurality of side ports 134 (e.g., the first side port 134A and the second side port 134B) are located in the elongate cannula 150 in a distal chamber 151 thereof that is immediately proximal to the closed distal end 133. More particularly, the elongate cannula 150 of the elongate hollow stylet 132 defines an outer side wall 152 that surrounds an outer lumen 153 of the elongate hollow stylet 132. In addition, located within the outer lumen 153 of the elongate cannula 150 is a first inner side wall 154 that surrounds a middle lumen 155, and a first passageway is defined between the first inner side wall 154 and the outer side wall 152. Further, located within the middle lumen 155 of the elongate cannula 150 is a second inner side wall 156 that surrounds an inner lumen 157, and a second passageway is defined between the first inner side wall 154 and the second inner side wall 156, as well as a third passageway inside the second inner side wall 156. That is, the spacing and arrangement of the outer side wall 152, the first inner side wall 154, and the second inner side wall 156 define three separate passageways therein, each having a cross sectional size that gets smaller as one traverses from the outer side wall 152 inward. In some embodiments, the middle lumen 155 may be concentric with the outer lumen 153 and/or the inner lumen 157. A first distal opening 153-1 of the outer lumen 153, a second distal opening 155-1 of the middle lumen 155, and a third distal opening 157-1 of the inner lumen 157 are all open to the distal chamber 151 of the elongate hollow stylet 132. A technical effect is thus realized in which each of the lumens 153, 155, 157 maintains a separation of fluids until the fluids reach the distal chamber 151. For example, a first component disposed within the middle lumen 155 and a second component disposed within the inner lumen 157 do not mix together until the components reach the distal chamber 151, thereby avoiding a situation where the components prematurely mix, as described herein.

Still referring to FIGS. 1 and 4, in some embodiments, the plurality of side ports 134 radially extend (e.g., extend in a circumferential direction) from the distal chamber 151 and through the outer side wall 152 of the elongate hollow stylet 132 at the distal end 132-2 thereof. The closed stylet needle tip 135 is defined, at least in part, by the closed distal end 133 of the elongate hollow stylet 132. The closed stylet needle tip 135 is attached (e.g., welded, press fit, or with adhesive) to the elongate cannula 150 to distally close the distal chamber 151 of the elongate cannula 150 of the elongate hollow stylet 132. That is, the closed stylet needle tip 135 terminates a distal extent of the distal chamber 151 of the elongate hollow stylet 132. When the delivery system 100 is assembled, the plurality of side ports 134 are in fluid communication with the plurality of output ports 112A-1, 112B-1 of the dual chamber applicator 110 by way of the plurality of input ports 132A-1, 132B-1, the middle lumen 155, the inner lumen 157, and the distal chamber 151 of the elongate hollow stylet 132. In addition, the plurality of side ports 134 are in fluid communication with the auxiliary input port 132C-1 by way of the outer lumen 153 and the distal chamber 151 of the elongate hollow stylet 132.

Referring to FIG. 1 and with reference to the embodiment of FIG. 5, the elongate hollow stylet 132 may be constructed, for example, by an elongate cannula 150 being fixedly connected to the closed distal end 133′. As particularly shown in FIG. 5, the plurality of side ports 134 (e.g., the first side port 134A and the second side port 134B) are located in the elongate cannula 150 in a distal chamber 151 thereof that is immediately proximal to the closed distal end 133′. More particularly, the elongate cannula 150 of the elongate hollow stylet 132 defines an outer side wall 152 that surrounds an outer lumen 153 of the elongate hollow stylet 132. In addition, located within the outer lumen 153 of the elongate cannula 150 is a first inner side wall 154 that surrounds a middle lumen 155, the middle lumen 155 being concentric with the outer lumen 153, and a first passageway is defined between the first inner side wall 154 and the outer side wall 152. Further, located within the middle lumen 155 of the elongate cannula 150 is a second inner side wall 156 that surrounds an inner lumen 157, the inner lumen 157 being concentric with the outer lumen 153 and the middle lumen 155 and a second passageway is defined between the first inner side wall 154 and the second inner side wall 156, as well as a third passageway inside the second inner side wall 156. That is, the spacing and arrangement of the outer side wall 152, the first inner side wall 154, and the second inner side wall 156 define three separate passageways therein, each having a cross sectional size that gets smaller as one traverses from the outer side wall 152 inward. A first distal opening 153-1 of the outer lumen 153, a second distal opening 155-1 of the middle lumen 155, and a third distal opening 157-1 of the inner lumen 157 are all open to the distal chamber 151 of the elongate hollow stylet 132.

Still referring to FIGS. 1 and 5, in some embodiments, the plurality of side ports 134 radially extend from the distal chamber 151 and through the outer side wall 152 of the elongate hollow stylet 132 at the distal end 132-2 thereof. The closed distal end 133′ is defined, at least in part, by a distal wall 138 thereof. The distal wall 138 is attached (e.g., welded, press fit, or with adhesive) to the elongate cannula 150 or integrated with the outer side wall 152 (e.g., an extension of the outer side wall 152) to distally close the distal chamber 151 of the elongate cannula 150 of the elongate hollow stylet 132. That is, the distal wall 138 terminates a distal extent of the distal chamber 151 of the elongate hollow stylet 132. When the delivery system 100 is assembled, the plurality of side ports 134 are in fluid communication with the plurality of output ports 112A-1, 112B-1 of the dual chamber applicator 110 by way of the plurality of input ports 132A-1, 132B-1, the outer lumen 153, the inner lumen 157, and the distal chamber 151 of the elongate hollow stylet 132. In addition, the plurality of side ports 134 are in fluid communication with the auxiliary input port 132C-1 by way of the outer lumen 153 and the distal chamber 151 of the elongate hollow stylet 132.

In some aspects, as particularly shown in the embodiments of FIG. 4 and FIG. 5, the plurality of side ports 134 in the distal end 132-2 of elongate hollow stylet 132 includes a first side port 134A, a second side port 134B, and a third side port (not shown), which are located in the closed distal end 133, 133′ and arranged (e.g., in a ring pattern, around a perimeter of elongate hollow stylet 132) such as, for example, in 120 degree increments. In one application, for example, the plurality of side ports 134 include at least three side ports (e.g., 3 to 7 side ports) to have the at least three side ports near, but proximal to, the closed stylet needle tip 135 such that the flowable multi-component hydrogel may be delivered 360 degrees around elongate hollow stylet 132, so as to ensure spacer placement in an entirety of a target area. As another example, it is contemplated that in some applications it may be desirable to have the plurality of side ports 134 configured as two, or two pairs, of diametrically opposed side ports.

Optionally, it is further contemplated that the plurality of side ports 134 may include at least two longitudinally spaced side ports, such as for example, a side port longitudinally spaced (e.g. 1 to 3 millimeters) proximal to another side port. For example, the plurality of side ports 134 may include two rings of three side ports arranged around a perimeter of elongate hollow stylet 132, wherein the two rings of three side ports are longitudinally spaced in the distal end 132-2 of the elongate hollow stylet 132.

Referring to FIG. 1 and with reference to the embodiment of FIG. 6, the elongate hollow stylet 132 may be constructed, for example, by an elongate cannula 150 being fixedly connected to the open distal portion 137. As particularly shown in FIG. 6, a distal port 134C is defined within a distal wall 139 of the open distal portion 137 in the elongate cannula 150. The distal wall 139 defines a distal chamber 151 adjacent to the open distal portion 137. More particularly, the elongate cannula 150 of the elongate hollow stylet 132 defines an outer side wall 152 that is coupled to or integrated with the distal wall 138. The outer side wall 152 surrounds an outer lumen 153 of the elongate hollow stylet 132. In addition, located within the outer lumen 153 of the elongate cannula 150 is a first inner side wall 154 that surrounds a middle lumen 155, the middle lumen 155 being concentric with the outer lumen 153, and a first passageway is defined between the first inner side wall 154 and the outer side wall 152. Further, located within the middle lumen 155 of the elongate cannula 150 is a second inner side wall 156 that surrounds an inner lumen 157, the inner lumen 157 being concentric with the outer lumen 153 and the middle lumen 155 and a second passageway is defined between the first inner side wall 154 and the second inner side wall 156, as well as a third passageway inside the second inner side wall 156. That is, the spacing and arrangement of the outer side wall 152, the first inner side wall 154, and the second inner side wall 156 define three separate passageways therein, each having a cross sectional size that gets smaller as one traverses from the outer side wall 152 inward. A first distal opening 153-1 of the outer lumen 153, a second distal opening 155-1 of the middle lumen 155, and a third distal opening 157-1 of the inner lumen 157 are all open to the distal chamber 151 of the elongate hollow stylet 132.

Still referring to FIGS. 1 and 6, in some embodiments, the distal port 134C extends from the distal chamber 151 and through the distal wall 139 of the elongate hollow stylet 132. The open distal portion 137 is defined, at least in part, by the distal wall 139 thereof. The distal wall 139 is attached (e.g., welded, press fit, or with adhesive) to the elongate cannula 150 or integrated with the outer side wall 152 (e.g., an extension of the outer side wall 152) to at least partially close a distal end of the distal chamber 151 of the elongate cannula 150 of the elongate hollow stylet 132. That is, the distal wall 139 terminates a distal extent of the distal chamber 151 of the elongate hollow stylet 132. When the delivery system 100 is assembled, the distal port 134C is in fluid communication with the plurality of output ports 112A-1, 112B-1 of the dual chamber applicator 110 by way of the plurality of input ports 132A-1, 132B-1, the middle lumen 155, the inner lumen 157, and the distal chamber 151 of the elongate hollow stylet 132. In addition, the distal port 134C is in fluid communication with the auxiliary input port 132C-1 by way of the outer lumen 153 and the distal chamber 151 of the elongate hollow stylet 132.

In some aspects, the distal port 134C is positioned such that the flowable multi-component hydrogel may be delivered out the distal end 132-2 of the elongate hollow stylet 132, so as to ensure spacer placement in a targeted area. As another example, it is contemplated that in some applications it may be desirable to have a plurality of distal ports 134C located in the distal wall 139.

While not depicted in the embodiments of FIGS. 4-6, it is further contemplated that certain embodiments may include both side ports and a distal port. That is, certain embodiments of the elongate hollow stylet 132 may include one or more side ports 134 and a distal port 134C without departing from the scope of the present disclosure.

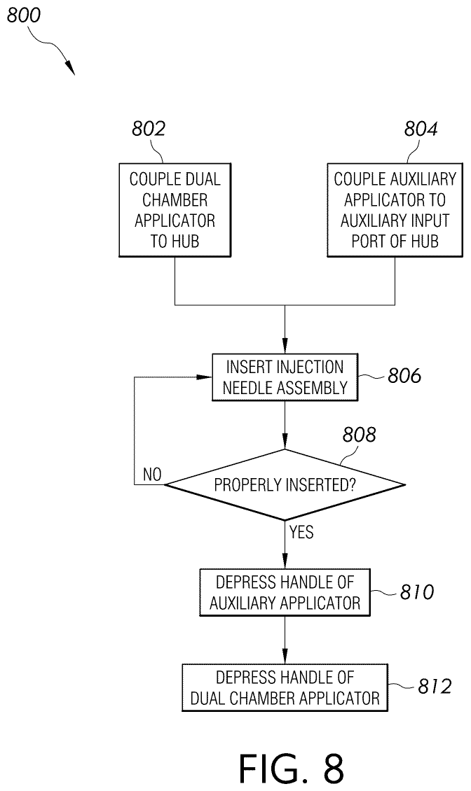

Referring now to FIGS. 1-2 and 7, the hub 131 may further include a plurality of passageways therein for fluidly coupling the first input port 132A-1 and the second input port 132B-1 to the distal chamber 151 depicted in the embodiments of FIGS. 4-6. Still referring to FIGS. 1-2 and 7, for example, in some aspects, a first channel 142A may couple the first input port 132A-1 to the middle lumen 155 by extending through the hub 131 from the first input port 132A-1 to the middle lumen 155. In another example, a second channel 142B may couple the second input port 132B-1 to the inner lumen 157 by extending through the hub 131 from the second input port 132B-1 to the inner lumen 157. In still another example, a third channel 142C may couple the auxiliary input port 132C-1 to the outer lumen 153 by extending through the hub 131 from the auxiliary input port 132C-1 to the outer lumen 153.

The positioning of the first channel 142A and the second channel 142B may be dependent on a location of the first input port 132A-1 and the second input port 132B-1. For example, in the aspect depicted in FIGS. 1, 2, and 7, the first input port 132A-1 and the second input port 132B-1 are spaced to be aligned with the output ports 112A-1, 112B-1 of the dual chamber applicator 110. As such, the first input port 132A-1 and the second input port 132B-1 are generally spaced the same distance radially outward from the center axis C3 of the injection needle assembly 130. In such an aspect, the first channel 142A may extend longitudinally toward the distal end 132-2 of the injection needle assembly 130 (e.g., parallel with the center axis C3 of the needle assembly) and couple to the middle lumen 155 near a distal end of the hub 131. The second channel 142B may extend at an angle with respect to the center axis C3 from the second input port 132B-1 to the inner lumen 157. However, it should be understood that the present disclosure is not limited to such. For example, in another aspect not depicted, the first channel 142A may have a first channel portion that extends longitudinally through the hub 131 and a second channel portion that extends laterally (e.g., transverse to the first channel portion) to fluidly couple the first input port 132A-1 to the middle lumen 155. The second input port 132B-1 may be located at the center of the hub 131 and the second channel 142B may extend longitudinally from the second input port 132B-1 to the inner lumen 157.

Unlike the first channel 142A and the second channel 142B, the positioning of the third channel 142C is not as dependent on a location of the auxiliary input port 132C-1. That is, the third channel 142 may define any fluid path between the auxiliary input port 132C-1 and the outer lumen 153 regardless of the location of the auxiliary input port 132C-1. For example, in the aspect depicted in FIGS. 1, 2, and 7, a first channel portion of the third channel 142C may extend laterally (e.g., transverse to the center axis C3) through the hub 131 and a second channel portion of the third channel 142C may extend longitudinally (e.g., transverse to the first channel portion) to fluidly couple the auxiliary input port 132C-1 to the outer lumen 153. In another example, the third channel 142C may extend at an angle between the auxiliary input port 132C-1 and the outer lumen 153. In yet another example, the third channel 142C may be an open cavity or the like between the auxiliary input port 132C-1 and the outer lumen 153 that is separated from the first channel 142A and the second channel 142B.

The auxiliary input port 132C-1 is depicted in FIGS. 1, 2, 3A-3B, and 7 as being located on a side of the body of the hub 131. That is, the auxiliary input port 132C-1 is depicted as being an opening in an outer wall of the hub 131. However, the location of the auxiliary input port 132C-1 is not limited by the present disclosure and may be positioned at any location on the hub 131. In some embodiments, the auxiliary input port 132C-1 may be positioned such that an object fluidly coupled thereto (e.g., connective tubing, the auxiliary applicator 120, or the like) can be coupled without hindering the coupling of the dual chamber applicator 110 to the hub 131, as described herein. In some embodiments, the auxiliary input port 132C-1 may be located within a protrusion or the like that is shaped, sized, and/or configured to couple to an object such as connective tubing, the auxiliary applicator 120, or the like.

In an embodiment, these features of the injection needle assembly 130 (e.g., the elongate hollow stylet 132 including the inner lumen 157, the middle lumen 155, the distal chamber 151, and the port(s) 134) may advantageously result in an assembly that separately delivers the separate components of the multi-component hydrogel to the point where the hydrogel is to be applied such that the components are not mixed together until they reach the point where the spacer is to be located, thereby avoiding issues relating to clogging or the like. Further, the features of the injection needle assembly 130 (e.g., the elongate hollow stylet 132 including the outer lumen 153, the distal chamber 151 and the port(s) 134) may advantageously result in an assembly where the same assembly can be used to distribute another fluid, such as saline a hydro-dissection solution, or the like, to a target site before or after distribution of the multi-component hydrogel without the need to insert a different device into the space, disconnect and connect different components, and/or the like.

While the present disclosure generally relates to the elongate cannula 150 having the outer lumen 153, the middle lumen 155, and the inner lumen 157 (e.g., 3 separate passageways), each of which is fluidly coupled to a respective port (e.g., the first input port 132A-1, the second input port 132B-1, and the auxiliary input port 132C-1, the present disclosure is not limited to such. That is, in some embodiments, the elongate cannula 150 may include two concentric lumens whereby one of the lumens is coupled to two of the ports (e.g., one lumen coupled to the first input port 132A-1 and the auxiliary input port 132C-1 while another lumen is coupled to the second input port 132B-1, one lumen coupled to the first input port 132B-1 while the other is coupled to the second input port 132B-1 and the auxiliary input port 132C-1). In such embodiments, the spacer material may be delivered via the same lumen as one of the two precursor materials since the spacer material is generally delivered at a different time as the precursor materials (e.g., before the precursor materials). Further, in such embodiments, the auxiliary input port 132C-1 may be capped off after delivery of the spacer material so that one of the precursor materials does not backflow through the auxiliary input port 132C-1. In other embodiments, the elongate cannula 150 may include two concentric lumens whereby a first one of the lumens is coupled to both of the first input port 132A-1 and the second input port 132B-1, and a second one of the lumens is coupled to the auxiliary input port 132C-1.

Referring to FIGS. 1, 2 and 7, in some embodiments, the hub 131 may have a connector that is shaped and sized to connect the hub 131 to the dual chamber applicator 110 and align the various ports thereof for the delivery of the precursor materials. For example, the hub 131 may have a quarter turn connector. In some aspects, the quarter turn connector of the hub 131 includes a circular recess 180 sized to receive a corresponding circular protrusion on the distal end 111-2 of the dual chamber applicator 110. The circular recess 180 is generally a recess that is defined by a wall 182 extending proximally (e.g., in the +x direction of the coordinate axes of FIG. 1) from a proximal end 130-1 of the injection needle assembly 130 (e.g., extending proximally from the hub 131 of the injection needle assembly 130). The wall 182 extends around the center axis C3 of the injection needle assembly 130 to form the circular recess 180. The wall 182 may be shaped and sized such that the circular recess 180 formed thereby corresponds to the shape and size of the circular protrusion of the dual chamber applicator 110. In some aspects, the wall 182 may be an extension of side walls of the hub 131 of the injection needle assembly 130.

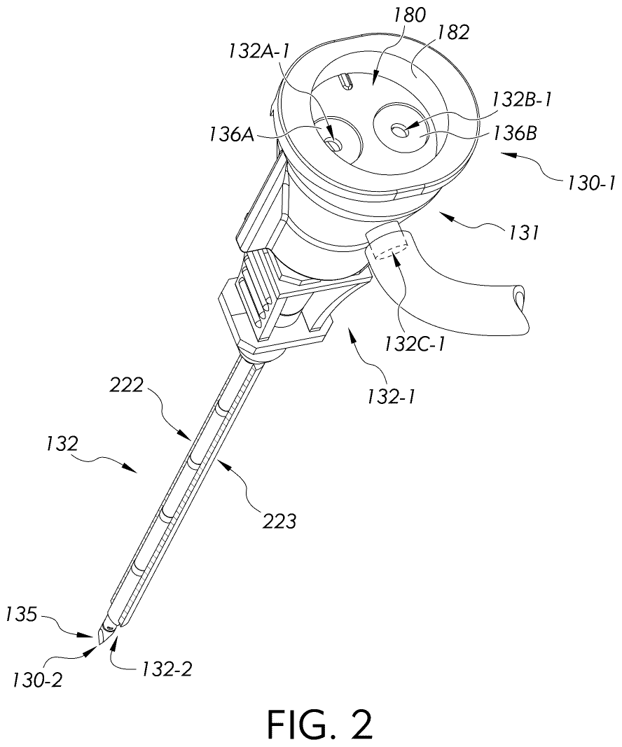

The plurality of input ports 132A-1, 132B-1 extend out of the proximal end 130-1 of the injection needle assembly 130 within the circular recess 180. That is, the circular recess 180 includes the plurality of input ports 132A-1, 132B-1 therein. In some aspects, the circular recess 180 may include one or more features (e.g., additional recesses, retention pieces, channels, etc.) that are adapted to hold at least one seal around the plurality of input ports 132A-1, 132B-1. For example, as particularly shown in FIGS. 1-2, a first seal 136A may be held within the circular recess 180 around the first input port 132A-1 and a second seal 136B may be held within the circular recess 180 around the second input port 132B-1. The seals 136A, 136B may each be any seal that allows the injection needle assembly 130 to form a seal with the dual chamber applicator 110 when brought together as described herein such that the first input port 132A-1 is joined and sealed with the first output port 112A-1 (e.g., to form a fluid coupling between the first input port 132A-1 and the first output port 112A-1) and the second input port 132B-1 is joined and sealed with the second output port 112B-1 (e.g., to form a fluid coupling between the second input port 132B-1 and the second output port 112B-1). For example, the seals may be O-rings, stadium shaped seals, oval seals, and/or the like. While a single seal is depicted herein for each port, the present disclosure is not limited to such. For example, a single seal, such as a figure eight shaped gasket or the like, may be used to individually seal the ports as described herein. However, it should be understood that the ports (e.g., the first input port 132A-1 and the second input port 132B-1) remain sealed from one another to avoid premature combining of components prior to reaching the distal chamber 151 (FIGS. 4-6). In some embodiments, one or more seal containment protrusions (not depicted) may be used to contain the seals 136A, 136B around the respective input ports 132A-1, 132B-1. That is, protrusions that extend proximally (e.g., in the +x direction of the coordinate axes of FIG. 1) from the hub 131 around a perimeter of each of the input ports 132A-1, 132B-1 may be shaped and sized so that the seals are press fit within the protrusions. In some embodiments, the seals 136A, 136B may be affixed via an adhesive or the like around the respective input ports 132A-1, 132B-1. In some embodiments, the seals 136A, 136B may be coupled to or integrated with the dual chamber applicator 110 instead of the hub 131.

It should be appreciated that the circular recess, the corresponding protrusion, and the quarter turn connector are merely illustrative examples. That is, the present disclosure is not limited to these features as a means for coupling the hub 131 to the dual chamber applicator 110 and other components that achieve a similar purpose (e.g., interlocking tabs, clips, screw threads, etc.) are also contemplated and included within the scope of the present disclosure.

Referring to FIGS. 1, 2, and 3A-3B, the injection needle assembly 130 may be used in conjunction with the introducer cannula 160 (also sometimes referred to in the art as a coaxial introducer needle) to allow elongate hollow stylet 132 to be removed from the introducer cannula 160, while maintaining access to the procedure site with the coaxial introducer needle. That is, a portion of the delivery system 100 may be removed from the introducer cannula 160 and replaced with a variety of other instruments, such as a biopsy device or another stylet. In some aspects, the introducer cannula 160 has a coaxial hub 162, a coaxial cannula 164, a cannula lumen 166 and a distal annular rim 168. The cannula lumen 166, for example, is configured (e.g., having a cylindrical shape) to receive the elongate hollow stylet 132 of the injection needle assembly 130. When the elongate hollow stylet 132 of the injection needle assembly 130 is fully inserted into the cannula lumen 166 of the introducer cannula 160 (e.g., distal movement of the elongate hollow stylet 132 is stopped by contact of the hub 131 of the injection needle assembly 130 with the coaxial hub 162 of the introducer cannula 160, the plurality of side ports 134 of the elongate hollow stylet 132 are located distal to the distal annular rim 168 of the introducer cannula 160. That is, the distal end 132-2 of the elongate hollow stylet 132 extends beyond the distal annular rim 168 such that the introducer cannula 160 does not block the plurality of side ports 134.

Turning now to FIG. 8 and with reference to FIGS. 1-7, a method 800 of using the delivery system 100 described herein generally includes, for example, coupling the dual chamber applicator 110 (which is pre-filled with the precursor material of the multi-component hydrogel) to the hub 131 at block 802. In addition, the auxiliary applicator 120 (which is pre-filled with a fluid such as saline, a hydro-dissection solution, or the like), is coupled to the auxiliary input port 132C-1 at block 804. The injection needle assembly 130 is then inserted into a target region in a subject, such as, for example, between a prostate and a rectal wall of a subject at block 806. In some embodiments, it may be confirmed that the distal end 132-2 is appropriately positioned (decision block 808). If it is not appropriately positioned, the process may return to block 806 for further insertion and movement. If it is appropriately positioned, the process moves to blocks 810 and 812 where, materials are dispensed within the target region. In one particular embodiment, the handle 125 of the auxiliary applicator 120 is depressed at block 810 to move the piston 124 to distribute the fluid within the auxiliary applicator 120, which travels via the outer lumen 153 to the distal chamber 151 and out of the injection needle assembly 130 via the port(s) 134 into the target region to create a space (e.g., a space between a prostate and a rectum of a subject). Thereafter, the handle 115 is depressed at block 812 to move the pistons 114A, 114B to distribute the precursor materials within the dual chamber applicator 110, which travel via the middle lumen 155 and the inner lumen 157, respectively, to the distal chamber 151 where they mix and flow out of the injection needle assembly 130 via the port(s) 134 into the space created by the fluid to form the spacer. It should be appreciated that the order of distribution of materials in blocks 810 and 812 is merely illustrative for one particular procedure, and another order is contemplated.

It should now be understood that the present disclosure relates to various multi-component hydrogel delivery systems that include components that deliver a multi-component hydrogel along an access path, to a target area, such as a space between a prostate and a rectum. The delivery systems described herein include components that deliver the precursor materials for the multi-component hydrogel, which includes a dual chamber applicator. In addition, the delivery systems described herein further include an injection needle assembly and an auxiliary syringe used to deliver the hydro-dissecting solution. The delivery systems described herein include a hub coupled to an elongate hollow stylet, where the hub includes a pair of input ports for coupling to the dual chamber applicator and an auxiliary port for coupling to the auxiliary syringe, and the elongate hollow stylet includes a mixing chamber at a distal end thereof, an inner lumen disposed concentrically within a middle lumen that is concentrically disposed within an outer lumen to define three separate passageways, two of which extend from the two chambers of the applicator and the third from the auxiliary port to the mixing chamber at the distal end.

The following embodiments also relate to the present disclosure:

In an embodiment, a needle assembly comprises a hub comprising first and second input ports and an auxiliary port; and an injection needle assembly comprising: an elongate hollow stylet extending distally from the hub, the elongate hollow stylet having a proximal portion at the hub and a distal portion spaced a distance from the proximal portion, the elongate hollow stylet comprising: an outer side wall extending from the proximal portion to the distal portion and defining an outer lumen that is fluidly coupled to the auxiliary port, a first inner side wall extending from the proximal portion to the distal portion and defining a middle lumen disposed within the outer lumen such that the middle lumen is concentric with the outer lumen and has a cross-sectional size that is smaller than the outer lumen, the middle lumen being fluidly coupled to the first input port, a second inner side wall extending from the proximal portion to the distal portion and defining an inner lumen disposed within the middle lumen such that the inner lumen is concentric with the outer lumen and the middle lumen and has a cross-sectional size that is smaller than the middle lumen, the inner lumen being fluidly coupled to the second input port, and a mixing chamber disposed at the distal portion of the elongate hollow stylet, the mixing chamber fluidly coupled to the outer lumen, the middle lumen, and the inner lumen.

The needle assembly according to any of the previous embodiments, wherein the mixing chamber at the distal portion of the elongate hollow stylet comprises at least one side port arranged around a perimeter of the elongate hollow stylet.

The needle assembly according to any of the previous embodiments, wherein the mixing chamber at the distal portion of the elongate hollow stylet comprises at least two longitudinally spaced side ports in the distal portion arranged around a perimeter of the elongate hollow stylet.

The needle assembly according to any of the previous embodiments, wherein the mixing chamber at the distal portion of the elongate hollow stylet comprises at least three side ports in the distal portion arranged around a perimeter of the elongate hollow stylet.

The needle assembly according to any of the previous embodiments, wherein the mixing chamber comprises at least one tip port located at the distal portion of the injection needle assembly.

The needle assembly according to any of the previous embodiments, wherein the distal end of the elongate hollow stylet is a blunt tip that terminates a distal extent of the inner lumen, the middle lumen, and the outer lumen.

The needle assembly according to any of the previous embodiments, wherein the injection needle assembly comprises a closed stylet needle tip.

The needle assembly according to any of the previous embodiments, wherein the hub further comprises at least one seal disposed around the first and second input ports.

The needle assembly according to any of the previous embodiments, wherein the at least one seal is an O-ring or a stadium shaped seal.

The needle assembly according to any of the previous embodiments, wherein the hub further comprises one or more seal containment protrusions disposed radially outward from the first and second input ports.

The needle assembly according to any of the previous embodiments, wherein the hub comprises a connector shaped and sized to connect the hub to a dual chamber applicator.

The needle assembly according to any of the previous embodiments, wherein the connector is a quarter turn connector.

The needle assembly according to any of the previous embodiments, wherein the connector is further shaped and sized to align the first and second input ports with output ports of the dual chamber applicator.

The needle assembly according to any of the previous embodiments, wherein the auxiliary port is shaped and sized to be coupled to an auxiliary applicator.

In another embodiment, a fluid delivery system comprises a dual chamber applicator comprising two chambers separate from one another, each chamber comprising at least one output port on a distal end thereof; and the injection needle assembly according to any one the previous embodiments.

The fluid delivery system according to any of the previous embodiments, further comprising an auxiliary applicator fluidly coupled to the injection needle assembly via the auxiliary port.

In another embodiment, a fluid delivery system comprises a dual chamber applicator comprising two chambers separate from one another, each chamber comprising at least one output port on a distal end thereof; and an injection needle assembly comprising: a hub comprising: first and second input ports aligned with the at least one output port of each of the two chambers of the dual chamber applicator, and an auxiliary port; and an elongate hollow stylet extending distally from the hub, the elongate hollow stylet having a proximal portion at the hub and a distal portion spaced a distance from the proximal portion, the elongate hollow stylet comprising: an outer side wall extending from the proximal portion to the distal portion and defining an outer lumen that is fluidly coupled to the auxiliary port, a first inner side wall extending from the proximal portion to the distal portion and defining a middle lumen disposed within the outer lumen such that the middle lumen is concentric with the outer lumen and has a cross-sectional size that is smaller than the outer lumen, the middle lumen being fluidly coupled to the first input port, a second inner side wall extending from the proximal portion to the distal portion and defining an inner lumen disposed within the middle lumen such that the inner lumen is concentric with the outer lumen and the middle lumen and has a cross-sectional size that is smaller than the middle lumen, the inner lumen being fluidly coupled to the second input port, and a mixing chamber disposed at the end distal portion of the elongate hollow stylet, the mixing chamber fluidly coupled to the outer lumen, the middle lumen, and the inner lumen.

The fluid delivery system according to any of the previous embodiments, further comprising an auxiliary applicator fluidly coupled to the injection needle assembly via the auxiliary port.

In another embodiment, a fluid delivery system comprises an injection needle assembly having a proximal end and a distal end spaced a length from the proximal end, the injection needle assembly comprising a hub disposed at the proximal end, the hub comprising: a first input port fluidly coupled to a first lumen extending the length of the injection needle assembly, a second input port fluidly coupled to a second lumen extending the length of the injection needle assembly, the second lumen separated from the first lumen, and an auxiliary port fluidly coupled to a third lumen extending the length of the injection needle assembly, the third lumen separated from the first lumen and the second lumen; a dual chamber applicator comprising: a first chamber having a first output port aligned with the first input port of the hub, a second chamber separate from the first chamber, the second chamber having a second output port aligned with the second input port of the hub; and an auxiliary applicator fluidly coupled to the auxiliary port of the hub.

In another embodiment, a method of delivering fluids to a subject, the method comprising: inserting a distal end of an injection needle assembly of a fluid delivery system into a target area of the subject, wherein the fluid delivery system comprises a dual chamber applicator and an auxiliary applicator fluidly coupled via a hub and an elongate hollow stylet defining three separate fluid paths to a distal mixing chamber disposed at the end distal portion of the elongate hollow stylet; causing a first fluid within the auxiliary applicator to be delivered to the target area via a first one of the three separate fluid paths to create a space; and causing two precursor materials within the dual chamber applicator to be delivered to the space via a second and a third one of the three separate fluid paths and mixed, wherein mixing of the two precursor materials results in a hydrogel being formed in the space.

The method according to any of the previous embodiments, further comprising removing the injection needle assembly from the target area.

In another embodiment, a needle assembly comprising: a hub comprising first and second input ports and an auxiliary port; and an injection needle assembly comprising: an elongate hollow stylet extending distally from the hub, the elongate hollow stylet having a proximal portion at the hub and a distal portion spaced a distance from the proximal portion, the elongate hollow stylet comprising: an outer side wall extending from the proximal portion to the distal portion and defining an outer lumen that is fluidly coupled to the auxiliary port, a first inner side wall extending from the proximal portion to the distal portion and defining a middle lumen disposed within the outer lumen such that the outer lumen surrounds the middle lumen, the middle lumen being fluidly coupled to the first input port, a second inner side wall extending from the proximal portion to the distal portion and defining an inner lumen disposed within the middle lumen such that the middle lumen surrounds the inner lumen, the inner lumen being fluidly coupled to the second input port, and a mixing chamber disposed at the distal portion of the elongate hollow stylet, the mixing chamber fluidly coupled to the outer lumen, the middle lumen, and the inner lumen.

In another embodiment, a needle assembly that isolates components to be mixed until the components reach a target area, the needle assembly comprising: a hub comprising first and second input ports and an auxiliary port; and an injection needle assembly comprising: an elongate hollow stylet extending distally from the hub, the elongate hollow stylet having a proximal portion at the hub and a distal portion spaced a distance from the proximal portion, the elongate hollow stylet comprising: an outer side wall extending from the proximal portion to the distal portion and defining an outer lumen that is fluidly coupled to the auxiliary port, a first inner side wall extending from the proximal portion to the distal portion and defining a middle lumen disposed within the outer lumen such that the outer lumen surrounds the middle lumen, the middle lumen being fluidly coupled to the first input port, a second inner side wall extending from the proximal portion to the distal portion and defining an inner lumen disposed within the middle lumen such that the middle lumen surrounds the inner lumen, the inner lumen being fluidly coupled to the second input port, and a mixing chamber disposed at the distal portion of the elongate hollow stylet, the mixing chamber fluidly coupled to the outer lumen, the middle lumen, and the inner lumen and configured to mix fluids received from the middle lumen and the inner lumen.

In another embodiment, a fluid delivery system, comprising: a dual chamber applicator comprising two chambers separate from one another, each chamber comprising at least one output port on a distal end thereof; and an injection needle assembly comprising: a hub comprising: first and second input ports aligned with the at least one output port of each of the two chambers of the dual chamber applicator, and an auxiliary port; and an elongate hollow stylet extending distally from the hub, the elongate hollow stylet having a proximal portion at the hub and a distal portion spaced a distance from the proximal portion, the elongate hollow stylet comprising: an outer side wall extending from the proximal portion to the distal portion and defining an outer lumen that is fluidly coupled to the auxiliary port, a first inner side wall extending from the proximal portion to the distal portion and defining a middle lumen disposed within the outer lumen such that the outer lumen surrounds the middle lumen, the middle lumen being fluidly coupled to the first input port, a second inner side wall extending from the proximal portion to the distal portion and defining an inner lumen disposed within the middle lumen such that the middle lumen surrounds the inner lumen, the inner lumen being fluidly coupled to the second input port, and a mixing chamber disposed at the end distal portion of the elongate hollow stylet, the mixing chamber fluidly coupled to the outer lumen, the middle lumen, and the inner lumen.

In another embodiment, a fluid delivery system, comprising: a dual chamber applicator comprising two chambers separate from one another, each chamber comprising at least one output port on a distal end thereof; and an injection needle assembly that isolates components to be mixed until the components reach a target area, the needle assembly comprising: a hub comprising: first and second input ports aligned with the at least one output port of each of the two chambers of the dual chamber applicator, and an auxiliary port; and an elongate hollow stylet extending distally from the hub, the elongate hollow stylet having a proximal portion at the hub and a distal portion spaced a distance from the proximal portion, the elongate hollow stylet comprising: an outer side wall extending from the proximal portion to the distal portion and defining an outer lumen that is fluidly coupled to the auxiliary port, a first inner side wall extending from the proximal portion to the distal portion and defining a middle lumen disposed within the outer lumen such that the outer lumen surrounds the middle lumen, the middle lumen being fluidly coupled to the first input port, a second inner side wall extending from the proximal portion to the distal portion and defining an inner lumen disposed within the middle lumen such that the middle lumen surrounds the inner lumen, the inner lumen being fluidly coupled to the second input port, and a mixing chamber disposed at the end distal portion of the elongate hollow stylet, the mixing chamber fluidly coupled to the outer lumen, the middle lumen, and the inner lumen and configured to mix fluids received from the middle lumen and the inner lumen.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A needle assembly, comprising:
a hub comprising first and second input ports, an auxiliary port, and a connector shaped and sized to connect the hub to a dual chamber applicator; and
an injection needle assembly comprising:
an elongate hollow stylet extending distally from the hub, the elongate hollow stylet having a proximal portion at the hub and a distal portion spaced a distance from the proximal portion, the elongate hollow stylet comprising:
an outer side wall extending from the proximal portion to the distal portion and defining an outer lumen that is fluidly coupled to the auxiliary port,
a first inner side wall extending from the proximal portion to the distal portion and defining a middle lumen disposed within the outer lumen such that the outer lumen surrounds the middle lumen, the middle lumen being fluidly coupled to the first input port,
a second inner side wall extending from the proximal portion to the distal portion and defining an inner lumen disposed within the middle lumen such that the middle lumen surrounds the inner lumen, the inner lumen being fluidly coupled to the second input port, and
a mixing chamber disposed at the distal portion of the elongate hollow stylet, the mixing chamber fluidly coupled to the outer lumen, the middle lumen, and the inner lumen,
wherein the connector aligns the first and second input ports with output ports of the dual chamber applicator.

2. The needle assembly according to claim 1, wherein the mixing chamber at the distal portion of the elongate hollow stylet comprises at least one side port extending in a circumferential direction of the elongate hollow stylet.

3. The needle assembly according to claim 1, wherein the mixing chamber at the distal portion of the elongate hollow stylet comprises at least two longitudinally spaced side ports in the distal portion that extend in a circumferential direction of the elongate hollow stylet.

4. The needle assembly according to claim 1, wherein the mixing chamber at the distal portion of the elongate hollow stylet comprises at least three side ports in the distal portion that extend in a circumferential direction of the elongate hollow stylet.

5. The needle assembly according to claim 1, wherein the mixing chamber comprises at least one tip port located at the distal portion of the injection needle assembly.

6. The needle assembly according to claim 1, wherein the distal end of the elongate hollow stylet is a blunt tip that terminates a distal extent of the inner lumen, the middle lumen, and the outer lumen.

7. The needle assembly according to claim 1, wherein the injection needle assembly comprises a closed stylet needle tip.

8. The needle assembly according to claim 1, wherein the hub further comprises at least one seal disposed around the first and second input ports.

9. The needle assembly according to claim 8, wherein the at least one seal is an O-ring or a stadium shaped seal.

10. The needle assembly according to claim 1, wherein the hub further comprises one or more seal containment protrusions disposed radially outward from the first and second input ports.

11. (canceled)

12. The needle assembly according to claim 1, wherein the connector is a quarter turn connector.

13. (canceled)

14. The needle assembly according to claim 1, wherein the auxiliary port is shaped and sized to be coupled to an auxiliary applicator.

15. The needle assembly according to claim 1, wherein the outer lumen is concentric with at least one of the middle lumen and the inner lumen.

16. The needle assembly according to claim 1, wherein the middle lumen is concentric with the inner lumen.

17. (canceled)

18. (canceled)

19. A fluid delivery system, comprising:
a dual chamber applicator comprising two chambers separate from one another, each chamber comprising at least one output port on a distal end thereof; and
an injection needle assembly comprising:
a hub comprising:
first and second input ports aligned with the at least one output port of each of the two chambers of the dual chamber applicator,
an auxiliary port, and
a connector shaped and sized to connect the hub to the dual chamber applicator; and
an elongate hollow stylet extending distally from the hub, the elongate hollow stylet having a proximal portion at the hub and a distal portion spaced a distance from the proximal portion, the elongate hollow stylet comprising:
an outer side wall extending from the proximal portion to the distal portion and defining an outer lumen that is fluidly coupled to the auxiliary port,
a first inner side wall extending from the proximal portion to the distal portion and defining a middle lumen disposed within the outer lumen such that the outer lumen surrounds the middle lumen, the middle lumen being fluidly coupled to the first input port,
a second inner side wall extending from the proximal portion to the distal portion and defining an inner lumen disposed within the middle lumen such that the middle lumen surrounds the inner lumen, the inner lumen being fluidly coupled to the second input port, and
a mixing chamber disposed at the end distal portion of the elongate hollow stylet, the mixing chamber fluidly coupled to the outer lumen, the middle lumen, and the inner lumen,
wherein the connector aligns the first and second input ports with each of the plurality of output ports of the dual chamber applicator.

20. The fluid delivery system according to claim 19, further comprising an auxiliary applicator fluidly coupled to the injection needle assembly via the auxiliary port.

21. A fluid delivery system, comprising:
an injection needle assembly having a proximal end and a distal end spaced a length from the proximal end, the injection needle assembly comprising a hub disposed at the proximal end, the hub comprising:
a first input port fluidly coupled to a first lumen extending the length of the injection needle assembly,
a second input port fluidly coupled to a second lumen extending the length of the injection needle assembly, the second lumen separated from the first lumen, and
an auxiliary port fluidly coupled to a third lumen extending the length of the injection needle assembly, the third lumen separated from the first lumen and the second lumen;
a dual chamber applicator comprising:
a first chamber having a first output port aligned with the first input port of the hub,
a second chamber separate from the first chamber, the second chamber having a second output port aligned with the second input port of the hub; and
an auxiliary applicator fluidly coupled to the auxiliary port of the hub,
wherein the injection needle assembly is couplable to the dual chamber applicator via a connector in the hub, the connector shaped and sized to align the first and second input ports with the first and second output ports.

22. (canceled)

23. (canceled)
